SEQUENCE LISTING (4 sequences)

SEQ ID NO: 1 - DNA, 4711 bp, Artificial Sequence
  Synthetic  Developed expression cassette which contains the CMV promoter, optimized sequence of the S protein of SARS-CoV-2, and polyadenylation signal
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 2 - DNA, 5984 bp, Artificial Sequence
  Synthetic  Developed expression cassette which contains the CAG promoter, optimized sequence of the S protein of SARS-CoV-2, and polyadenylation signal
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctccccccctcccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcgggggggctgcgaggggaacaaaggctgcgtgcggggtgtgtgcgtggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcggggggacggggcagggcggggttcggcttctggcgtgtgaccggcggctctagaaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagatttttttatattttgttttgtgttattttttctttaacatccctaaaattttccttacatgttttactagccagatttttcctcctctcctgactactcccagtcatagctgtccctcttctcttatggagatc

SEQ ID NO: 3 - DNA, 5314 bp, Artificial Sequence
  Synthetic  Developed expression cassette which contains the EF1 promoter, optimized sequence of the S protein of SARS-CoV-2, and polyadenylation signal
ggtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcacaagcttggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatctagagtcggggcggccggccgctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatccgagtgtcgataag

SEQ ID NO: 4 - DNA, 4678 bp, Artificial Sequence
  Synthetic  Developed expression cassette which contains the CMV promoter, optimized sequence of the S protein of SARS-CoV-2, and polyadenylation signal
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccatgtttgtgttccttgtgttattgccactagtctctagtcagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatattgccgataccacagacgccgtacgagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgccaccactgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgacatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgacatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctcaaaggagtcaaattacattacacataagatatcgcggccgctcgagtctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

(19) United States
(12) Patent Application Publication
(10) Pub. No.: US 2022/0249655 A1
(43) Pub. Date: Aug. 11, 2022

(54) AGENT FOR INDUCING SPECIFIC IMMUNITY AGAINST SEVERE ACUTE RESPIRATORY SYNDROME VIRUS SARS-COV-2 IN LYOPHILIZED FORM (VARIANTS)

(71) Applicant: Federal State Budgetary Institution "National Research Centre for Epidemiology And Microbilogy Named, Moscow (RU)

(72) Inventors: Olga Vadimovna Zubkova, Khimki (RU); Tatiana Andreevna Ozharovskaia, Korolev (RU); Inna Vadimovna Dolzhikova, Lobnya (RU); Olga Popova, Ufa (RU); Dmitrii Viktorovich Shchebliakov, Moscow (RU); Daria Mikhailovna Grousova, Moscow (RU); Alina Shahmirovna Dzharullaeva, Moscow (RU); Amir Ildarovich Tukhvatulin, Moscow (RU); Natalia Mikhailovna Tukhvatulina, Moscow (RU); Dmitrii Nikolaevich Shcherbinin, Moskovskaya oblast (RU); Ilias Bulatovich Esmagambetov, Dmitrov (RU); Elizaveta Alexsandrovna Tokarskaya, Moscow (RU); Andrei Gennadevich Botikov, Moscow (RU); Alina Sergeevna Eroxova, Bryansk (RU); Fatima Magometovna Izhaeva, Karahaevo-Cherkesskaya Respublika (RU); Natalya Anatolevna Nikitenko, Moscow (RU); Nadezhda Leonidovna Lubenets, Moskovskaya oblast (RU); Aleksandr Sergeevich Semikhin, Moscow (RU); Sergey Vladimirovich Borisevich, Sergiev Posad (RU); Boris Savelievich Naroditsky, Moscow (RU); Denis Yuryevich Logunov, Moscow (RU); Aleksandr Leonidovich Gintsburg, Moscow (RU)

(21) Appl. No.: 17/715,945

(22) Filed: Apr. 7, 2022

Related U.S. Application Data
(63) Continuation of application No. PCT/RU2021/000182, filed on Apr. 30, 2021.

(30) Foreign Application Priority Data
Feb. 10, 2021 (RU) 2021103101

(51) Int. Cl. A61K 39/215; A61K 9/19; A61K 47/02; A61K 47/26; A61K 47/18; A61K 9/00; A61P 31/14

(52) U.S. Cl. CPC A61K 39/215; A61K 9/19; A61K 47/02; A61K 47/26; A61K 2039/5256; A61K 9/0043; A61K 9/0019; A61P 31/14; A61K 47/183

(57) ABSTRACT

The invention relates to a biomolecule agent for inducing specific immunity against severe acute respiratory syndrome virus SARS-CoV-2, in lyophilized (freeze-dried) form, which contains a single active component, comprising the expression vector including either the genome of the recombinant strain of human adenovirus serotype 26 or 5, wherein the E1 and E3 regions are deleted, and an integrated expression cassette selected from SEQ ID NO:1, SEQ ID NO:2, or SEQ ID NO:3; or the genome of the recombinant strain of simian adenovirus serotype 25, wherein the E1 and E3 regions are deleted, and an integrated expression cassette is selected from SEQ ID NO:4, SEQ ID NO:2, or SEQ ID NO:3. The genome of the recombinant strain of human adenovirus serotype 26 may include the ORF6-Ad26 region replaced by ORF6-Ad5.

A buffer solution for reconstitution of the lyophilized form of the agent may contain the following, mass %: tris from 0.0180-0.0338; sodium chloride from 0.1044-0.1957; sucrose from 5.4688-10.2539; magnesium chloride hexahydrate from 0.0015-0.0028; EDTA from 0.0003-0.0005; polysorbate-80 from 0.0037-0.0070; and water to fill.

The agent can be administered via intranasal and/or intramuscular routes. The invention promotes humoral and cell-mediated immune responses against SARS-CoV-2 virus among broad strata of the population.

### CROSS-REFERENCE TO RELATED APPLICATIONS

The present application is a continuation application of International Application No. PCT/RU2021/00182, filed Apr. 30, 2021, which claims priority to Russian Patent Application No. 2021103101, filed on Feb. 10, 2021, the contents of both applications are hereby incorporated by reference in their entirety.

### INCORPORATION BY REFERENCE-SEQUENCE LISTING

This application includes an electronically submitted sequence listing in .txt format. The .txt file contains a sequence listing entitled “110620_00392_SequenceListing.txt” which was created on Apr. 7, 2022 and is 28,616 bytes in size. The sequence listing contained in this .txt file is part of the specification and is hereby incorporated by reference herein in its entirety.

### FIELD OF THE INVENTION

The invention relates to biotechnology, immunology and virology. The claimed agent can be used for the prevention of diseases caused by severe acute respiratory syndrome virus SARS-CoV-2.

### BACKGROUND OF THE INVENTION

At the end of 2019, an outbreak of a newly emerging infection was recorded in the People's Republic of China, with the epicenter in Wuhan, the provincial capital of Hubei. Later on, it was found out that the infection was caused by an earlier unknown coronavirus named as SARS-CoV-2. Within several months SARS-CoV-2 has spread around the world and become pandemic affecting over 200 countries. By Feb. 1, 2021 the number of cases was more than 103 million and above 2 million people died.

The coronavirus infection is transmitted through the following main modes: respiratory droplets (or dust particles) and contact. The mean incubation period is 5-6 days and then initial symptoms of the disease appear. The usual signs of COVID-19 include fever, dry cough, shortness of breath, and fatigue. A sore throat, joint pain, runny nose, and headache have been also reported as less common symptoms. However, clinical course of the disease is characterized by varying severity from asymptomatic cases to severe acute respiratory syndrome and death.

Rapid geographic spread of SARS-CoV-2 and high mortality rates have caused an urgent need to develop effective agents for the prevention of diseases caused by this virus. Thus, currently the development of safe and effective vaccines for SARS-CoV-2 is recognized as a global top priority.

Within a year after the pandemic onset, multiple pharma companies proposed their variants of COVID-19 vaccine candidates.

Pfizer pharmaceutical company in partnership with BioNTech biocompany developed a vaccine known as BNT162b2 (tozinameran). It is based on modified mRNA encoding a mutant S protein of SARS-CoV-2 embedded in lipid nanoparticles. The vaccination regimen requires two injections spaced 21 days apart (F. P. Polack et al. Safety and Efficacy of the BNT162b2 mRNA Covid-19 Vaccine. N Engl J Med 2020; 383: 2603-2615).

Moderna pharmaceutical company and the United States National Institute of Allergy and Infectious Diseases (NIAID) co-developed the mRNA-1273 vaccine. Its active component is mRNA encoding a mutant S protein of SARS-CoV-2 coated in lipid shell. According to the immunization regimen, the vaccine is to be given as two doses 28 days apart (L. A. Jackson et al. An mRNA Vaccine against SARS-CoV-2—Preliminary Report. N Engl J Med 2020; 383:1920-1931).

The University of Oxford in collaboration with AstraZeneca plc developed a viral vectored vaccine ChAdOx1 nCoV-19 (AZD1222). Its active component is a chimpanzee adenovirus ChAdOx1 encoding a codon-optimized full-length S protein sequence of the SARS-CoV-2 virus (GenBank MN908947) with a human tissue plasminogen activator leader sequence. According to the immunization regimen, the vaccine is to be given as two doses 28 days apart (M. Voysey et al. Safety and efficacy of the ChAdOx1 nCoV-19 vaccine (AZD1222) against SARS-CoV-2: an interim analysis of four randomised controlled trials in Brazil, South Africa, and the UK. The Lancet. Vol. 397, Issue 10269, P99-111, 2021).

CanSino developed a viral vectored vaccine against COVID-19 based on a replication incompetent human adenovirus Type 5 (Ad5), expressing the SARS-CoV-2 full-length S glycoprotein. It is a one-dose regimen vaccine. (GenBank YP_009724390) (Feng-Cai Zhu et al. Immunogenicity and safety of a recombinant adenovirus type-5-vectored COVID-19 vaccine in healthy adults aged 18 years or older: a randomised, double-blind, placebo-controlled, phase 2 trial. The Lancet. Vol. 369, Issue 10249, P479-488, 2020).

Research teams at the Janssen Pharmaceutical Companies of Johnson & Johnson in cooperation with Beth Israel Deaconess Medical Center using Janssen's AdVac® technology platform have developed several vaccine candidates. Based on the results of the safety and efficacy studies, a vaccine candidate Ad26.COV2.S (Ad26COVS1) was selected. The vaccine is based on recombinant E1/E3-deleted adenovirus serotype 26 vector containing the SARS-CoV-2 virus S protein gene, with the mutation of a furin cleavage site and two stabilizing praline mutations. Now, two immunization regimens are tested: the vaccine is given as a single dose or two doses 8 weeks apart (J. Sadoff et al. Interim Results of a Phase 1-2a Trial of Ad26.COV2.S Covid-19 Vaccine. N Engl J Med, 2021 Jan. 13. DOI: 10.1056/NEJMoa2034201).

Thus, it should be noted that the vast majority of COVID-19 vaccines require a two-shot regimen.

Each of the above mentioned vaccines has its advantages and limitations. Thus, mRNA vaccines have less severe side effects. However, they are less immunogenic compared with viral vectored vaccines. Besides, RNA is more fragile and sensitive to storage conditions.

Recombinant viral-vectored vaccines achieve high immunogenicity. But the disadvantage of vaccines of this class is a potential induction of the immune response to the vector portion which makes revaccination more difficult. In addition, adenoviruses are circulating in the human population and therefore some people may have pre-existing immunity against these viruses. Expression vectors based on other mammalian adenoviruses are used to resolve the pre-existing immunity issue, but such vectors have a lower ability to enter human cells, which, in turn, reduces the efficacy of vaccines.

There is a technical solution according to patent RF No. 2731342 (published on 1 Sep. 2020) chosen as a prototype by the authors of the claimed invention. The following variants of a pharmaceutical agent for inducing specific immunity against severe acute respiratory syndrome virus SARS-CoV-2 are known from this patent:
which contains component 1, comprising an agent in the form of expression vector based on the genome of recombinant human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3, and which also contains component 2, comprising an agent in the form of expression vector based on the genome of recombinant human adenovirus serotype 5, wherein the E1 and E3 regions are deleted, with an integrated expression cassette selected from SEQ ID NO:1, SEQ ID NO:2, SEQ ID NO:3
which contains component 1, comprising an agent in the form of expression vector based on the genome of recombinant human adenovirus serotype 26, wherein the E1 and E3 regions are deleted, and the ORF6-Ad26 region is replaced by ORF6-Ad5, with an integrated expression cassette selected from SEQ ID NO:1, SEQ ID NO:2, SEQ ID NO:3, and which also contains component 2, comprising an agent in the form of expression vector based on the genome of recombinant simian adenovirus serotype 25, wherein the E1 and E3 regions are deleted, with an integrated expression cassette selected from SEQ ID NO:4, SEQ ID NO:2, SEQ ID NO:3.
which contains component 1, comprising an agent in the form of expression vector based on the genome of recombinant simian adenovirus serotype 25, wherein the E1 and E3 regions are deleted, with an integrated expression cassette selected from SEQ ID NO:4, SEQ ID NO:2, SEQ ID NO:3, and which also contains component 2, comprising an agent in the form of expression vector based on the genome of recombinant human adenovirus serotype 5, wherein the E1 and E3 regions are deleted, with an integrated expression cassette selected from SEQ ID NO:1, SEQ ID NO:2, SEQ ID NO:3.

Also, the patent discloses the administration of the above mentioned variants of agents for inducing specific immunity against the severe acute respiratory syndrome SARS-CoV-2 virus, wherein component 1 and component 2 are used in an effective amount, sequentially, with a time interval of at least one week.

It should be pointed out that this mode of administration has several drawbacks. Thus, for example, each of the components of the pharmaceutical agent may cause side effects and allergic reactions; therefore in case of using a two-shot vaccination regimen the number of such events will increase. Besides, such immunization regimen is associated with multiple practical difficulties, as it is necessary to ensure that patients are present for getting the second dose after a certain time interval. In addition, there are numerous logistical challenges linked to a timely delivery of the necessary agent components.

Thus, field of the invention elicits a need for expanding a range of pharmaceutical agents able to induce immune response to the SARS-CoV-2 virus among broad strata of the population.

The technical aim of the claimed group of inventions is to create agents containing a single active component and along with this ensuring the effective induction of immune response to the SARS-CoV-2 virus among broad strata of the population.

### DISCLOSURE OF THE INVENTION

Solution of the technical problem is a variant of the agent for inducing specific immunity against severe acute respiratory syndrome virus SARS-CoV-2 in lyophilized (freeze-dried) form which contains, as a single active component, the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3.

Also, there is created a variant of the agent for inducing specific immunity against severe acute respiratory syndrome virus SARS-CoV-2 in lyophilized (freeze-dried) form which contains, as a single active component, the expression vector based on the genome of the recombinant strain of human adenovirus serotype 5, wherein the E1 and E3 regions are deleted, with an integrated expression cassette selected from SEQ ID NO:1, SEQ ID NO:2, SEQ ID NO:3.

Further, there is claimed a variant of the agent for inducing specific immunity against severe acute respiratory syndrome virus SARS-CoV-2 in lyophilized (freeze-dried) form which contains, as a single active component, the expression vector based on the genome of recombinant simian adenovirus serotype 25, wherein the E1 and E3 regions are deleted, with an integrated expression cassette selected from SEQ ID NO:4, SEQ ID NO:2, SEQ ID NO:3.

At that, for the particular case of implementation, a buffer solution of the agent for the reconstituted lyophilized (freeze-dried) form contains the following, mass %:

| | |
|---|---|
| tris | from 0.0180 to 0.0338 |
| sodium chloride | from 0.1044 to 0.1957 |
| sucrose | from 5.4688 to 10.2539 |
| magnesium chloride hexahydrate | from 0.0015 to 0.0028 |
| EDTA | from 0.0003 to 0.0005 |
| polysorbate-80 | from 0.0037 to 0.0070 |
| water | the remaining part. |

Each of the agent variants is used for inducing specific immunity against the severe acute respiratory syndrome SARS-CoV-2 virus.

With that, the agent is intended for intranasal or intramuscular administration. Also, the agent can be administered concomitantly and simultaneously via intranasal and intramuscular routes.

At that, for the particular case of implementation, the agent is administered via intranasal route in a dose from 5*10¹⁰ to 5*10¹¹ viral particles; via intramuscular route—in a dose from 5*10¹⁰ to 5*10¹¹ viral particles. And for the concomitant administration via intranasal and intramuscular routes, a dose from 5*10¹⁰ to 5*10¹¹ viral particles is administered intramuscularly and a dose from 5*10¹⁰ to 5*10¹¹ viral particles is administered intranasally.

The concomitant administration envisages intranasal and intramuscular administration within a single vaccination procedure.

The technical result is the creation of an agent which ensures the development of humoral and cell-mediated immune responses to the SARS-Cov-2 virus among broad strata of the population.

The main goal of immunization is to ensure the effective and long-lasting protection against the pathogen. One of the ways for achieving this goal is to use multi-dose vaccine series. When the human body is exposed to a vaccine antigen for the first time, the activation of the two main components of the adaptive immune response occurs, namely B lymphocytes and effector T lymphocytes.

Following activation, B lymphocytes are transformed into plasma cells responsible for antibody production, and also converted into memory B cells. Effector T lymphocytes are divided into two major types: helper T cells (CD4+) and cytotoxic (killer) T cells (CD8+). The key function of helper T cells is to promote the development of the humoral and cellular immune responses. The main function of cytotoxic T cells is to kill damaged cells of the host. Killer T cells are considered one of the essential components of the anti-viral immune response. However, following immunization the numbers of antigen-specific immune cells decrease with time, and so a booster dose of the vaccine is administered. The latter enables the immune system to maintain the appropriate numbers of antigen-specific T- and B cells (required to ensure the body's protection against pathogens).

The development of a single-component agent which will induce sustainable immune response after a single-shot immunization regimen is a complicated research and practical task. However, it is difficult to overestimate the significance of such development. A single-dose vaccine administration can promote higher rates of mass immunization that are critical in the pandemic conditions. Also, this agent could be beneficial for the emergency use and immunization of mobile groups of people (migrant tribes, etc.). Further, it is worth noting that the administration of a single-dose agent is associated with less adverse events in humans, such as injury rates and numbers of side effects and allergic reactions.

The advantages of the developed agent also include the storage of its lyophilized (freeze-dried) form at temperatures of +2° C. and +8° C. (in contrast to the liquid form stored at below-freezing temperatures) which guarantees convenient storage and transportation.

### BRIEF DESCRIPTION OF THE FIGURES

FIG. 1 illustrates the results of assessing the humoral immune response to SARS-CoV-2 virus antigen in volunteers immunized with lyophilized (freeze-dried) form of the developed agent according to variant 1,

Y-axis—IgG titer against the RBD of the S glycoprotein of SARS-CoV-2.

X-axis—days.

[symbol] IgG titer against the RBD of the S glycoprotein of SARS-CoV-2 in each of the volunteers involved in the study, at Day 14

[symbol] IgG titer against the RBD of the S glycoprotein of SARS-CoV-2 in each of the volunteers involved in the study, at Day 21

[symbol] IgG titer against the RBD of the S glycoprotein of SARS-CoV-2 in each of the volunteers involved in the study, at Day 28

Geometric mean of antibody titers is depicted as a black line for each of the data groups. The statistically significant difference between the values at days 14, 21 and 28 is shown by a bracket, above which p-value for the Wilcoxon T test is indicated.

FIG. 2 illustrates the results assessing the humoral immune response to SARS-CoV-2 virus antigen in volunteers immunized with lyophilized (freeze-dried) form of the developed agent according to variant 2,

Y-axis—IgG titer against the RBD of the S glycoprotein of SARS-CoV-2.

X-axis—days.

[symbol] IgG titer against the RBD of the S glycoprotein of SARS-CoV-2 in each of the volunteers involved in the study, at Day 14

[symbol] IgG titer against the RBD of the S glycoprotein of SARS-CoV-2 in each of the volunteers involved in the study, at Day 21

[symbol] IgG titer against the RBD of the S glycoprotein of SARS-CoV-2 in each of the volunteers involved in the study, at Day 28

Geometric mean of antibody titers is depicted as a black line for each of the data groups. The statistically significant difference between the values at days 14, 21 and 28 is shown by a bracket, above which p-value for the Wilcoxon T test is indicated.

FIG. 3 illustrates the results of assessing the immunization efficacy in volunteers who received lyophilized form of the developed agent according to variant 1, as estimated by the percentage of proliferating CD8+ (A) and CD4+ (B) lymphocytes re-stimulated by S antigen of SARS-CoV-2.

Y-axis—the number of proliferating cells, %

X-axis—days.

[symbol] —symbol used to denote the percentage of proliferating CD8+ in each of the volunteers at Day 0.

[symbol] —symbol used to denote the percentage of proliferating CD8+ in each of the volunteers at Day 14.

[symbol] —symbol used to denote the percentage of proliferating CD8+ in each of the volunteers at Day 28.

[symbol] —symbol used to denote the percentage of proliferating CD4+ in each of the volunteers at Day 0.

[symbol] —symbol used to denote the percentage of proliferating CD4+ in each of the volunteers at Day 14.

[symbol] —symbol used to denote the percentage of proliferating CD4+ in each of the volunteers at Day 28.

Median value is depicted as a black line for each of the data groups. The statistically significant difference between the values obtained at days 0, 14 and 28 is shown by a bracket and symbols *, p<0.05; **, p<0.01; ****, p<0.001 (Mann-Whitney test).

FIG. 4 illustrates the results of assessing the immunization efficacy in volunteers who received lyophilized (freeze-dried) form of the developed agent according to variant 2, as estimated by the percentage of proliferating CD8+ (A) and CD4+ (B) lymphocytes re-stimulated by S antigen of SARS-CoV-2.

Y-axis—the number of proliferating cells, %

X-axis—days.

[symbol] —symbol used to denote the percentage of proliferating CD8+ in each of the volunteers at Day 0.

[symbol] —symbol used to denote the percentage of proliferating CD8+ in each of the volunteers at Day 14.

[symbol] —symbol used to denote the percentage of proliferating CD8+ in each of the volunteers at Day 28.

[symbol] —symbol used to denote the percentage of proliferating CD4+ in each of the volunteers at Day 0.

[symbol] —symbol used to denote the percentage of proliferating CD4+ in each of the volunteers at Day 14.

[symbol] —symbol used to denote the percentage of proliferating CD4+ in each of the volunteers at Day 28.

Median value is depicted as a black line for each of the data groups. The statistically significant difference between the values obtained at days 0, 14 and 28 is shown by a bracket and symbols *, p<0.05; **, p<0.01; ****, p<0.001 (Mann-Whitney test).

### IMPLEMENTATION OF THE INVENTION

The active component of the developed agent comprises an expression vector based on the genome of recombinant adenovirus strain with an integrated expression cassette containing a gene of SARS-CoV-2 antigen.

Adenoviral vectors can enter many different human cell types, ensure high levels of target antigen expression and assist in eluding both the humoral and cell-mediated immune responses. The FSBI “N. F. Gamaleya NRCEM” of the Ministry of Health of the Russian Federation has developed the following 3 variants of expression vectors based on the mammalian adenoviruses:
expression vector based on the genome of the recombinant strain of human adenovirus serotype 26, wherein the E1 and E3 regions are deleted, and the ORF6-Ad26 region is replaced by ORF6-Ad5
expression vector based on the genome of the recombinant strain of human adenovirus serotype 5, wherein the E1 and E3 regions are deleted
expression vector based on the genome of the recombinant strain of simian adenovirus serotype 25, wherein the E1 and E3 regions are deleted, with an integrated expression cassette selected from SEQ ID NO:4, SEQ ID NO:2, SEQ ID NO:3.

The SARS-CoV-2 virus surface S protein was selected as an antigen. It is one of the most promising antigens capable of inducing a strong and long-lasting immune response. It was also demonstrated that antibodies against the S protein of SARS-CoV-2 had virus neutralizing activity.

To maximize the induced immune response, the authors developed multiple variants of expression cassettes containing the S protein gene.

Expression cassette SEQ ID NO:1 contains the CMV promoter, SARS-CoV-2 virus S protein gene, and polyadenylation signal. The CMV promoter is a promoter of immediate early genes of cytomegalovirus that ensures constitutive expression in multiple cell types. However, a target-gene expression strength controlled by the CMV promoter varies for different cell types. Further, the level of transgene expression under CMV promoter control was shown to decline as the duration of cell cultivation increases. It occurs due to the suppression of gene expression relating to DNA methylation [Wang W., Jia Y L., Li Y C., Jing C Q., Guo X., Shang X F., Zhao C P., Wang T Y. Impact of different promoters, promoter mutation, and an enhancer on recombinant protein expression in CHO cells. /Scientific Reports—2017. —Vol. 8. —P. 10416]

Expression cassette SEQ ID NO:2 contains the CAG promoter, SARS-CoV-2 virus S protein gene, and polyadenylation signal. The CAG promoter is a synthetic promoter containing early enhancer of the CMV promoter, chicken β-actin promoter and chimeric intron (chicken β-actin and rabbit β-globin). Experiments demonstrated that the CAG promoter has a higher transcriptional activity compared to the CMV promoter [Yang C. Q., Li X. Y., Li Q., Fu S. L., Li H., Guo Z. K., Lin J. T., Zhao S. T. Evaluation of three different promoters driving gene expression in developing chicken embryo by using in vivo electroporation. /Genet. Mol. Res. —2014. —Vol. 13. —P. 1270-1277].

Expression cassette SEQ ID NO:3 contains the EF1 promoter, SARS-CoV-2 virus S protein gene, and polyadenylation signal. The EF1 promoter is a promoter of human eukaryotic translation elongation factor 1α (EF-1α). The promoter is constitutively active in a variety of cell types [Wang X, Xu Z, Tian Z, Zhang X, Xu D, Li Q, Zhang J, Wang T. The EF-1α promoter maintains high-level transgene expression from episomal vectors in transfected CHO-K1 cells. J Cell Mol Med. 2017 November; 21(11):3044-3054. doi: 10.1111/jcmm.13216. Epub 2017 May 30. PMID: 28557288; PMCID: PMC5661254.]. The EF-1α gene encodes the elongation factor 1α which is one of the most frequent proteins in eukaryotic cells and shows expression almost in all mammalian cell types. The EF-1α promoter frequently demonstrates its activity in the cells where viral promoters are unable to facilitate the expression of controlled genes and in the cells where viral promoters are gradually extinguished.

Expression cassette SEQ ID NO:4 contains the CMV promoter, SARS-CoV-2 virus S protein gene, and polyadenylation signal.

Thus, as a result of the accomplished task, the following 3 variants of agent were developed.
1) Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3
2) Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 5, wherein the E1 and E3 regions are deleted, with an integrated expression cassette selected from SEQ ID NO:1, SEQ ID NO:2, SEQ ID NO:3.
3) Agent for inducing specific immunity against severe acute respiratory syndrome virus SARS-CoV-2, in lyophilized (freeze-dried) form, which contains a single active component, comprising the expression vector based on the genome of the recombinant strain of simian adenovirus serotype 25, wherein the E1 and E3 regions are deleted, with an integrated expression cassette selected from SEQ ID NO:4, SEQ ID NO:2, SEQ ID NO:3.

The implementation of the invention is proven by the following examples:

### Example 1. Production of an Active Component of the Agent for Inducing Specific Immunity Against Severe Acute Respiratory Syndrome Virus SARS-CoV-2 Based on the Genome of the Recombinant Strain of Human Adenovirus Serotype 26

At the first stage, the following 3 variants of expression cassettes were designed:
expression cassette SEQ ID NO:1 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Synthesis of SARS-CoV-2 virus S protein gene was performed by the “Eurogen” ZAO company (Moscow).

In order to derive a recombinant strain of human adenovirus serotype 26, the following two plasmids produced in the FSBI “N. F. Gamaleya NRCEM” of the Ministry of Health of the Russian Federation were used: plasmid pAd26-Ends carrying homology arms of the genome of human adenovirus serotype 26, and plasmid pAd26-too, carrying the genome of recombinant human adenovirus serotype 26 with the open reading frame ORF6 of human adenovirus serotype 5 and the deletion of the E1 and E3 regions.

At the first stage of work, genetic engineering techniques were used to obtain plasmids pAd26-Ends-CMV-S-CoV2, pAd26-Ends-CAG-S-CoV2, pAd26-Ends-EF1-S-CoV2 based on plasmid pAd26-Ends, containing expression cassettes SEQ ID NO:1, SEQ ID NO:2 or SEQ ID NO:3, respectively, as well as carrying homology arms of the genome of human adenovirus serotype 26. Then, the obtained plasmids were linearized by a unique hydrolysis site and each of the plasmids was mixed with the recombinant vector pAd26-too. As a result of the homologous recombination, plasmids pAd26-too-CMV-S-CoV2, pAd26-too-CAG-S-CoV2, pAd26-too-EF1-S-CoV2 were produced that carry the genome of recombinant human adenovirus serotype 26 with the open reading frame ORF6 of human adenovirus serotype 5 and the deletion of the E1 and E3 regions, with the expression cassette SEQ ID NO:1, SEQ ID NO:2 or SEQ ID NO:3, respectively.

At the next stage, plasmids pAd26-too-CMV-S-CoV2, pAd26-too-CAG-S-CoV2, pAd26-too-EF1-S-CoV2 were hydrolyzed with the specific restriction endonucleases to remove the vector part. The derived DNA products were used for the transfection of HEK293 cell culture.

As a result of the completed work, the following recombinant strains of human adenovirus serotype 26 were obtained: Ad26-too-CMV-S-CoV2, Ad26-too-CAG-S-CoV2, Ad26-too-EF1-S-CoV2. A similar scheme was used to produce a control strain of human adenovirus serotype 26: Ad26-too which did not contain the SARS-CoV-2 S protein gene.

Thus, an expression vector was obtained which contains the genome of recombinant human adenovirus serotype 26, wherein the E1 and E3 regions are deleted and ORF6-Ad26 region is replaced by ORF6-Ad5, with an integrated expression cassette selected from SEQ ID NO:1, SEQ ID NO:2, SEQ ID NO:3; the expression vector is an active component of the developed agent.

### Example 2. Production of an Active Component of the Agent for Inducing Specific Immunity Against Severe Acute Respiratory Syndrome Virus SARS-CoV-2 Based on the Genome of the Recombinant Strain of Human Adenovirus Serotype 5

Three variants of expression cassettes were also used in this effort:
expression cassette SEQ ID NO:1 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Synthesis of SARS-CoV-2 virus S protein gene was performed by the “Eurogen” ZAO company (Moscow).

In order to derive a recombinant strain of human adenovirus serotype5, the following two plasmids produced in the FSBI “N. F. Gamaleya NRCEM” of the Ministry of Health of the Russian Federation were used:
plasmid pAd5-Ends carrying the homology arms of the genome of adenovirus serotype 5 (one of the homology arms is a beginning portion of the genome of human adenovirus serotype 5 (from the left inverted terminal repeat to the E1 region) and the sequence of the viral genome including pIX protein. The other homology arm contains the nucleotide sequence located after the ORF3 E4 region through the end of the genome)
plasmid pAd5-too carrying the genome of recombinant human adenovirus serotype 5 wherein the E1 and E3 regions are deleted.

At the first stage of work, genetic engineering techniques were used to obtain plasmids pAd5-Ends-CMV-S-CoV2, pAd5-Ends-CAG-S-CoV2, pAd5-Ends-EF1-S-CoV2 based on plasmid pAd5-Ends. The produced plasmids contained expression cassettes SEQ ID NO:1, SEQ ID NO:2 [symbol] SEQ ID NO:3, respectively, as well as carrying homology arms of the genome of adenovirus serotype 5. Then, the obtained plasmids were linearized by a unique hydrolysis site and each of the plasmids was mixed with the recombinant vector pAd5-too. As a result of the homologous recombination, plasmids pAd5-too-CMV-S-CoV2, pAd5-too-CAG-S-CoV2, pAd5-too-EF1-S-CoV2 were produced that carry the genome of recombinant human adenovirus serotype 5, wherein the E1 and E3 regions are deleted, with the expression cassette SEQ ID NO:1, SEQ ID NO:2 or SEQ ID NO:3, respectively.

At the next stage, plasmids pAd5-too-CMV-S-CoV2, pAd5-too-CAG-S-CoV2, pAd5-too-EF1-S-CoV2 were hydrolyzed with the specific restriction endonucleases to remove the vector part. The derived DNA products were used for the transfection of HEK293 cell culture.

As a result of the completed work, the following recombinant strains of human adenovirus serotype 5 were obtained: Ad5-too-CMV-S-CoV2, Ad5-too-CAG-S-CoV2, Ad5-too-EF1-S-CoV2. A similar scheme was used to produce a control strain of human adenovirus serotype 5: Ad5-too which did not contain the SARS-CoV-2 S protein gene.

Thus, an expression vector was obtained which contains the genome of recombinant human adenovirus serotype 5, wherein the E1 and E3 regions are deleted, with an integrated expression cassette selected from SEQ ID NO:1, SEQ ID NO:2, SEQ ID NO:3; the expression vector is an active component of the developed agent.

### Example 3. Production of an Active Component of the Agent for Inducing Specific Immunity Against Severe Acute Respiratory Syndrome Virus SARS-CoV-2 Based on the Genome of the Recombinant Strain of Simian Adenovirus Serotype 25

The following three variants of the expression cassettes were used in this effort:
expression cassette SEQ ID NO:4 contains the CMV promoter, SARS-CoV-2 virus S protein gene, and polyadenylation signal;
expression cassette SEQ ID NO:2 contains the CAG promoter, SARS-CoV-2 virus S protein gene, and polyadenylation signal;
expression cassette SEQ ID NO:3 contains the EF1 promoter, SARS-CoV-2 virus S protein gene, and polyadenylation signal.

Synthesis of SARS-CoV-2 virus S protein gene was performed by the “Eurogen” ZAO company (Moscow).

In order to obtain a recombinant strain of simian adenovirus serotype 25, the following two plasmids produced in the FSBI “N. F. Gamaleya NRCEM” of the Ministry of Health of the Russian Federation were used:
plasmid pSim25-Ends carrying the homology arms of the genome of simian adenovirus serotype 25
plasmid pSim25-null carrying the genome of recombinant simian adenovirus serotype 25 with the deletion of the E1 and E3 regions.

At the first stage of work, genetic engineering techniques were used to obtain plasmids p-Sim25-Ends-CMV-S-CoV2, p-Sim25-Ends-CAG-S-CoV2, p-Sim25-Ends-EF1-S-CoV2 based on pSim25-Ends. The produced plasmids contained expression cassettes SEQ ID NO:4, SEQ ID NO:2 or SEQ ID NO:3, respectively, as well as carrying homology arms of the genome of simian adenovirus serotype 25. Then, the obtained plasmids were linearized by a unique hydrolysis site and each of the plasmids was mixed with the recombinant vector pSim25-too. As a result of the homologous recombination, plasmids pSim25-too-CMV-S-CoV2, pSim25-too-CAG-S-CoV2, pSim25-too-EF1-S-CoV2 were produced that carry the genome of recombinant simian adenovirus serotype 25, wherein the E1 and E3 regions are deleted, with the expression cassette SEQ ID NO:4, SEQ ID NO:2 or SEQ ID NO:3, respectively.

At the next stage, plasmids pSim25-too-CMV-S-CoV2, pSim25-too-CAG-S-CoV2, pSim25-too-EF1-S-CoV2 were hydrolyzed with the specific restriction endonucleases to remove the vector part. The derived DNA products were used for the transfection of HEK293 cell culture.

As a result of the completed work, the following recombinant strains of simian adenovirus serotype 25 were obtained: simAd25-too-CMV-S-CoV2, simAd25-too-CAG-S-CoV2, simAd25-too-EF1-S-CoV2. A similar scheme was used to produce a control strain of simian adenovirus serotype 25: simAd25-too which did not contain the SARS-CoV-2 S protein gene.

Thus, an expression vector was obtained which contains the genome of the recombinant strain of simian adenovirus serotype 25, wherein the E1 and E3 regions are deleted, with an integrated expression cassette selected from SEQ ID NO:4, SEQ ID NO:2, SEQ ID NO:3; the expression vector is an active component of the developed agent.

### Example 4. Development of a Buffer Solution

The inventors have selected a water-based buffer solution (i.e. the agent in lyophilized form contains the buffer solution after the reconstitution with water) which ensures the stability of recombinant adenovirus particles. Tris(hydroxymethyl)aminomethane (Tris) was added to the buffer for maintaining the solution pH value. The added sodium chloride was required for reaching the necessary ionic force and osmolarity. Sucrose was added as a cryoprotectant. Magnesium chloride hexahydrate was added as a source of bivalent cations; EDTA—as an inhibitor of free-radical oxidation; Polysorbate-80—as a source of surfactant; ethanol 95% —as an inhibitor of free-radical oxidation.

For estimating concentrations of the substances included in the composition of the buffer solution for lyophilized form of the pharmaceutical agent, several variants of experimental groups were produced (Table 1). One of the active components of the agent was added to each of the produced buffer solutions:
expression vector based on the genome of the recombinant strain of human adenovirus serotype 26, wherein the E1 and E3 regions are deleted and ORF6-Ad26 region is replaced by ORF6-Ad5, with an integrated expression cassette SEQ ID NO:1, (Ad26-too-CMV-S-CoV2, 1*10¹¹ viral particles).
expression vector based on the genome of the recombinant strain of human adenovirus serotype 5, wherein the E1 and E3 regions are deleted, with an integrated expression cassette SEQ ID NO:1 (Ad5-too-CMV-S-CoV2, 1*10¹¹ viral particles).
expression vector based on the genome of the recombinant strain of simian adenovirus serotype 25, wherein the E1 and E3 regions are deleted, with an integrated expression cassette SEQ ID NO:4 (simAd25-too-CMV-S-CoV2, 1*10¹¹ viral particles).

The obtained agents were lyophilized and stored at +2 and +8° C. for 3 months and then changes in the titers of the recombinant adenoviruses were assessed.

TABLE 1
Composition of experimental buffer solutions

| | Composition of buffer solution | | | | | | |
|---|---|---|---|---|---|---|---|
| | | | | Magnesium | | | |
| | | Sodium | | chloride | | | |
| Group | Tris | chloride | Sucrose | hexahydrate | EDTA | Polysorbate- | |
| No. | (mg) | (mg) | (mg) | (mg) | (mg) | 80 (mg) | Water |
| 1 | 0.1936 | 1.403 | 73.5 | 0.0204 | 0.0038 | 0.05 | to 1 ml |
| 2 | 0.363 | 1.403 | 73.5 | 0.0204 | 0.0038 | 0.05 | to 1 ml |
| 3 | 0.242 | 1.1224 | 73.5 | 0.0204 | 0.0038 | 0.05 | to 1 ml |
| 4 | 0.242 | 2.1045 | 73.5 | 0.0204 | 0.0038 | 0.05 | to 1 ml |
| 5 | 0.242 | 1.403 | 58.8 | 0.0204 | 0.0038 | 0.05 | to 1 ml |
| 6 | 0.242 | 1.403 | 110.25 | 0.0204 | 0.0038 | 0.05 | to 1 ml |
| 7 | 0.242 | 1.403 | 73.5 | 0.01632 | 0.0038 | 0.05 | to 1 ml |
| 8 | 0.242 | 1.403 | 73.5 | 0.0306 | 0.0038 | 0.05 | to 1 ml |
| 9 | 0.242 | 1.403 | 73.5 | 0.0204 | 0.00304 | 0.05 | to 1 ml |
| 10 | 0.242 | 1.403 | 73.5 | 0.0204 | 0.0057 | 0.05 | to 1 ml |
| 11 | 0.242 | 1.403 | 73.5 | 0.0204 | 0.0038 | 0.04 | to 1 ml |
| 12 | 0.242 | 1.403 | 73.5 | 0.0204 | 0.0038 | 0.075 | to 1 ml |
| 13 | 0.242 | 1.403 | 73.5 | 0.0204 | 0.0038 | 0.05 | to 1 ml |

The results of the performed experiment demonstrated that the titers of recombinant adenoviruses did not change after their storage for 3 months in the buffer solution for lyophilized form of the agent at temperatures of +2° C. and +8° C.

Thus, the developed buffer solution for lyophilized form of the vaccine ensures the stability of all components of the developed agent in the following range of active moieties:

Tris: from 0.0180 mass % to 0.0338 mass %;

Sodium chloride: from 0.1044 mass % to 0.1957 mass %;

Sucrose: from 5.4688 mass % to 10.2539 mass %;

Magnesium chloride hexahydrate: from 0.0015 mass % to 0.0028 mass %;

EDTA: from 0.0003 mass % to 0.0005 mass %;

Polysorbate-80: from 0.0037 mass % to 0.0070 mass %;

Solvent: the remaining part.

### Example 5. Production of an Agent for Inducing Specific Immunity Against Severe Acute Respiratory Syndrome Virus SARS-CoV-2 in Lyophilized (Freeze-Dried) Form

To produce a lyophilized formulation of the developed pharmaceutical agent with the possibility of long-term storage at a temperature range from 2 to 8° C., the buffer solution selected in Example 4 was used which was mixed with the relevant active component.

Three individual lyophilization cycles were performed with the mentioned formulation were performed using the earlier selected lyophilization program (Table 2).

TABLE 2
Lyophilization program

| | Time, | | Pressure, | |
|---|---|---|---|---|
| Phase | min | T, ° C. | mTor | Notes |
| 1 | 300 | −70.0 | — | Product freezing |
| 2 | 10 | −40.0 | 300 | Condenser is turned on; vacuum |
| | | | | buildup |
| 3 | 1950 | −40.0 | 200 | Gradual heat up during the phase |
| 4 | 1950 | −30.0 | 100 | Gradual heat up during the phase |
| 5 | 1550 | −20.0 | 100 | Gradual heat up during the phase |
| 6 | 30 | −10.0 | 100 | Gradual heat up during the phase |
| 7 | 30 | +0.0 | 100 | Gradual heat up during the phase |
| 8 | 10 | from +0.0 | 100 | Completion of drying |
| | | to +15.0 | | |

The following eligibility criteria were chosen for the lyophilized product: appearance—dry porous mass presented as a tablet formulation, whole or crumbled, in white or off-white color; weight loss on drying (residual moisture) —no more than 5%, reconstitution time (no more than 5 minutes), value of the specific activity of the final dosage form.

The indicators achieved after freeze drying satisfied the eligibility criteria.

Thus, the following agents were obtained:

1. Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3,

2. Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 5, wherein the E1 and E3 regions are deleted, with an integrated expression cassette selected from SEQ ID NO:1, SEQ ID NO:2, SEQ ID NO:3.

3. Agent for inducing specific immunity against severe acute respiratory syndrome virus SARS-CoV-2, in lyophilized (freeze-dried) form, which contains a single active component, comprising the expression vector based on the genome of the recombinant strain of simian adenovirus serotype 25, wherein the E1 and E3 regions are deleted, with an integrated expression cassette selected from SEQ ID NO:4, SEQ ID NO:2, SEQ ID NO:3.

### Example 6. Toxicity of the Developed Agent after its Single-Dose Intravenous and Intramuscular Administration (Acute Toxicity) to Mice

This study was conducted to assess the acute toxicity of:

Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, SEQ ID NO:3

Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 5, wherein the E1 and E3 regions are deleted, with an integrated expression cassette selected from SEQ ID NO:1, SEQ ID NO:2, SEQ ID NO:3

Agent for inducing specific immunity against severe acute respiratory syndrome virus SARS-CoV-2, in lyophilized (freeze-dried) form, which contains a single active component, comprising the expression vector based on the genome of the recombinant strain of simian adenovirus serotype 25, wherein the E1 and E3 regions are deleted, with an integrated expression cassette selected from SEQ ID NO:4, SEQ ID NO:2, SEQ ID NO:3.

Outbred male and female mice 6-8 weeks old with the weight of 18-20 g were used in the study.

Calculation of the agent dose was based on the immunizing dose (10⁸ v. p.), found in the preliminary experiment using the susceptible animal specie—Syrian golden hamsters. Doses for mice were calculated depending on their weight. The minimal dose selected for toxicology studies in mice was 10⁸ v. p. which is the most close to the therapeutic dose. The interspecies scaling factor was not used for dose conversion; the doses were recalculated directly based on body weight according to the WHO guidelines for vaccine preparations.

As a result, the following doses were selected for administering to mice in this experiment:

10⁸ v. p.—close to the effective dose (ED) for mice;

10⁹ v. p.—20 times higher ED for mice;

10¹⁰ v. p.—200 times higher ED for mice;

10¹¹ v. p.—2000 times higher ED for mice;

Thus, the following experimental animal groups were formed:

1) Ad26-too-CMV-S-CoV2, 1*10⁸ v. p., 20 mice;

2) Ad26-too-CMV-S-CoV2, 1*10⁹ v. p., 20 mice;

3) Ad26-too-CMV-S-CoV2, 1*10¹⁰ v. p., 20 mice;

4) Ad26-too-CMV-S-CoV2, 1*10¹¹ v. p., 20 mice;

5) Ad5-too-CMV-S-CoV2, 1*10⁸ v. p., 20 mice;

6) Ad5-too-CMV-S-CoV2, 1*10⁹ v. p., 20 mice;

7) Ad5-too-CMV-S-CoV2, 1*10¹⁰ v. p., 20 mice;

8) Ad5-too-CMV-S-CoV2, 1*10¹¹ v. p., 20 mice;

9) simAd25-too-CMV-S-CoV2, 1*10⁸ v. p., 20 mice;

10) simAd25-too-CMV-S-CoV2, 1*10⁹ v. p., 20 mice;

11) simAd25-too-CMV-S-CoV2, 1*10¹⁰ v. p., 20 mice;

12) simAd25-too-CMV-S-CoV2, 1*10¹¹ v. p., 20 mice;

13) placebo (buffer solution), 20 mice.

Physical examination of every animal was performed daily for 14 days to record the signs of intoxication and the number of dead animals.

The following parameters of functional state of the laboratory animals were recorded: activity, mobility, external appearance, the condition of hair, eyes, ears, teeth and limbs. The assessed physiological functions included breathing, salivation, saliva, urine, excreta.

All the animals survived during the experiment. Animals from all groups looked healthy, were actively eating the feed, had an adequate response to the stimuli and showed their interest in exploring the environment. The hair coat is thick, even and shining, and lies close to the body; no hair loss or fragility was found. The muscle tone was not characterized by hypertonicity. The outer ears have no crusts, inflammation signs or twitching. The tooth color is normal and the teeth are not broken. The mice were well-nourished and did not suffer malnourishment. The abdominal area is not enlarged. Smooth breathing, without difficulty. Salivation is normal. Urination, urine color, gastrointestinal system parameters, muscular tone, and reflexes are within the normal physiological range. The behavior of the experimental animals did not differ from the animal behavior in the control group.

At Day 14 of the experiment, the scheduled euthanasia of mice by cervical dislocation was performed. In the course of the study, no animals were found in critical condition with the signs of inevitable death. Also, no animal deaths were reported.

Complete necropsy of all animals was carried out. The necropsy comprised the assessment the animal's body condition, inner surfaces and tracts, intracranial, thoracic, abdominal and pelvic cavities including the internal organs and tissues of these cavities, the neck with its organs and tissues, and the skeletomuscular system.

Gross postmortem examination did not reveal any effects of the agent on the internal organs of mice. Differences between the control and experimental groups of animals were not found. The weight gain did not differ between the control and experimental groups of animals.

### Example 7

Assessment of the Efficacy of Immunization with the Developed Agent Based on the Evaluation of Humoral Immune Response

One of the key characteristics of the efficacy of immunization is antibody titer. The example elicits the data relating to the changes in antibody titers against SARS-CoV-2 S protein at day 21 following the administration of the agent to laboratory animals.

The mammalian species—BALB/c mice, females weighing 18 g were used in the experiment. All animals were divided into 13 groups, 5 animals per group, to whom variants of the developed agent in lyophilized form were injected intramuscularly.

Water for injections in the amount of 1.0 ml was added to the vial containing the developed agent in lyophilized form, 10¹¹ viral particles/vial. Thus, the reconstituted lyophilized agent was obtained. Then, the vial was shaken up until the lyophilizate was fully dissolved, and 200 μl were injected intramuscularly to the animals.

The following groups of animals were formed:

1) Ad26-too-CMV-S-CoV2,

2) Ad26-too-CAG-S-CoV2,

3) Ad26-too-EF1-S-CoV2

4) Ad26-too

5) Ad5-too-CMV-S-CoV2,

6) Ad5-too-CAG-S-CoV2,

7) Ad5-too-EF1-S-CoV2

8) Ad5-too

9) simAd25-too-CMV-S-CoV2,

10) simAd25-too-CAG-S-CoV2,

11) simAd25-too-EF1-S-CoV2

12) simAd25-too

13) placebo (buffer)

Three weeks later, blood samples were taken from the tail vein of the animals, and the blood serum was separated. An enzyme-linked immunosorbent assay (ELISA) was used to measure antibody titers according to the following protocol:
1) Antigen was adsorbed onto wells of a 96-well ELISA plate for 16 hours at a temperature of +4° C.
2) Then, for preventing a non-specific binding, the plate was “blocked” with 5% milk dissolved in the blocking non-specific signal buffer in an amount of 100 μl per well. It was incubated in shaker at 37° C. for one hour.
3) Serum samples from the immunized mice were diluted 100-fold, and then a two-fold dilution series was prepared.
4) 50 μl of each of the diluted serum samples were added to the plate wells.
5) Then, incubation at 37° C. for 1 hour was performed.
6) After incubation the wells were washed three times with phosphate buffer.
7) Then, the secondary antibodies against mouse immunoglobulins conjugated with horseradish peroxidase were added.
8) Next, incubation at 37° C. for 1 hour was performed.
9) After incubation the wells were washed three times with phosphate buffer.
10) Then, tetramethylbenzidine (TMB) solution was added which is used as a substrate for horseradish peroxidase and is converted into a colored compound by the reaction. The reaction was stopped after 15 minutes by adding sulfuric acid. Next, using a spectrophotometer, the optical density (OD) of the solution was measured in each well at a wavelength of 450 nm.

Antibody titer was defined as the last dilution at which the optical density of the solution was significantly higher than in the negative control group. The obtained results (geometric mean) are presented in Table 3.

TABLE 3
Antibody titers against SARS-CoV-2 S protein in the blood
serum of mice (geometric mean of antibody titers)

| No. | Designation of animal group | Antibody titers |
|---|---|---|
| 1 | Ad26-too-CMV-S-CoV2, | 2111 |
| 2 | Ad26-too-CAG-S-CoV2, | 1838 |
| 3 | Ad26-too-EF1-S-CoV2 | 2111 |
| 4 | Ad26-too | 0 |
| 5 | Ad5-too-CMV-S-CoV2, | 38802 |
| 6 | Ad5-too-CAG-S-CoV2, | 33779 |
| 7 | Ad5-too-EF1-S-CoV2 | 25600 |
| 8 | Ad5-too | 0 |
| 9 | simAd25-too-CMV-S-CoV2, | 12800 |
| 10 | simAd25-too-CAG-S-CoV2, | 11143 |
| 11 | simAd25-too-EF1-S-CoV2 | 14703 |
| 12 | simAd25-too | 0 |
| 13 | placebo (buffer) | 0 |

Thus, the experimental results demonstrate that all the developed agents induce humoral immune response against SARS-CoV-2.

### Example 8. Evaluation of the Immunogenicity of the Developed Agent by Assessing Humoral Immune Response to the SARS-CoV-2 Virus Antigen in the Blood of Volunteers at Different Time Periods after Vaccination

The objective of this experiment was to determine the intensity of immune response to the SARS-CoV-2 virus antigen in the blood of volunteers at different time periods after vaccination with different variants of the developed agent.

Healthy volunteers 18-60 years of age were included in the trial. All participants of the trial were divided into several groups.

1) Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, 10¹¹ viral particles/dose, 9 individuals.

2) Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 5, wherein the E1 and E3 regions are deleted and the ORF6-Ad26 region is replaced by ORF6-Ad5, with an integrated expression cassette selected from SEQ ID NO:1, 10¹¹ viral particles/dose, 9 individuals.

Water for injections in an amount of 1.0 ml was added to the vial containing the developed agent in lyophilized form. Then, the vial was shaken up until the lyophilizate was fully dissolved. The developed agent was administered intramuscularly in the deltoid muscle (upper third of the outer aspect of the upper arm). In case where it was impossible to make injection in the deltoid muscle, the agent was injected in the lateral vastus muscle.

Blood samples were collected from the subjects prior to immunization and at days 14, 21, 28 and 42. The serum was separated from the obtained blood samples and used for determining antibody titers against the SARS-CoV-2 virus S antigen.

Antibody titer was measured using the test kit developed in the FSBI “N. F. Gamaleya NRCEM” of the Ministry of Health of the Russian Federation (RZN 2020/10393 2020 May 18) designed to determine IgG titer against the SARS-CoV-2 virus S protein RBD.

Plates with the preliminary adsorbed RBD (100 ng/well) was washed 5 times in washing buffer. Next, positive control (100 μl) and negative control (100 μl) in duplicates were added to the plate wells. A series of two-fold dilutions of the studied samples (two duplicates per sample) were added to the remaining plate wells. The plate was sealed with a film and incubated for 1 h at +37° C. while stirring at 300 rpm. Then, the wells were washed 5 times with working solution of the washing buffer. Next, 100 μl of working solution of the monoclonal antibody conjugate were added to each well, the plate was closed with an adhesive film and incubated for 1 h at +37° C. while stirring at 300 rpm. Then, the wells were washed 5 times with working solution of the washing buffer. Then, 100 μl of chromogenic substrate were added to each well and incubated for 15 minutes in a dark place at +20° C. After this step, the reaction was stopped by adding 50 μl of stop-reagent (1M solution of sulfuric acid) per well. The result was recorded within 10 min after stopping the reaction by measuring the optical density on spectrophotometer at a wavelength of 450 nm.

IgG titer was defined as a maximum serum dilution in which the value of OD450 in the serum of the immunized subject is twice higher than the value in the control serum (the subject's serum prior to immunization).

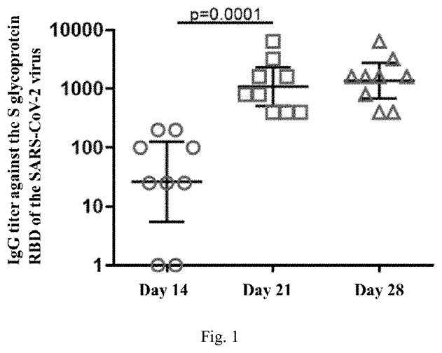

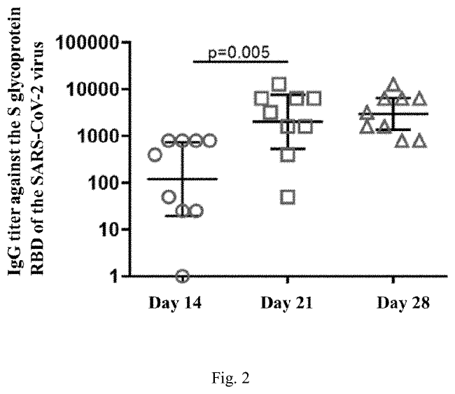

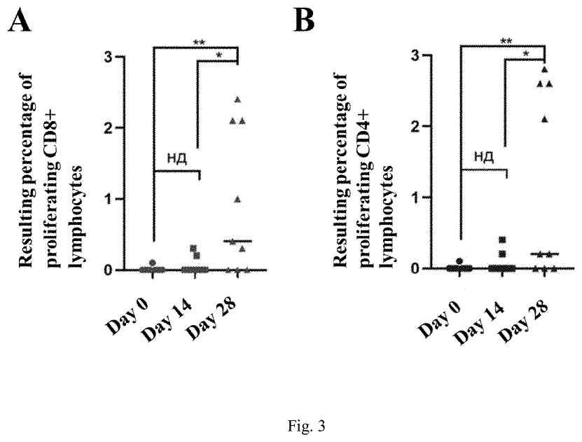

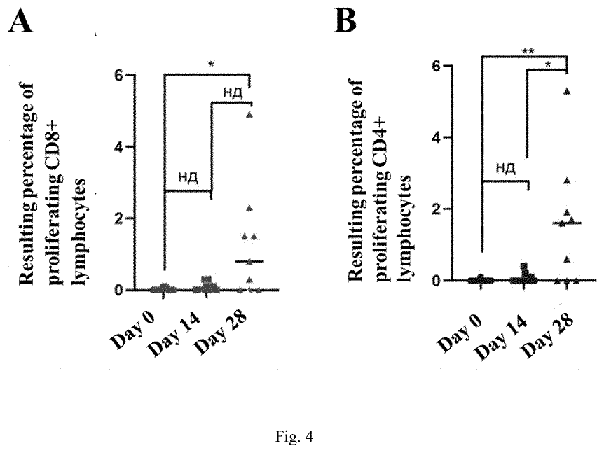

The results of assessment of the antibody titers against the SARS-CoV-2 antigen in the blood serum of volunteers after the administration of different variants of the developed agent are shown on FIG. 1, 2.

As demonstrated by the findings, the immunization of volunteers with both variants of the developed agent provides for achieving a strong (with a statistically significant difference from the values in the control, non-immunized group of volunteers) humoral immunity characterized by an increase in the antibody titer against the SARS-CoV-2 virus S protein. With that, the intensity of humoral immune response was growing as more days have passed since the date of immunization.

### Example 9. Evaluation of the Immunogenicity of the Developed Agent by Assessing Cell-Mediated Immune Response to the SARS-CoV-2 Virus Antigen in the Blood of Volunteers at Different Time Periods after Vaccination

The objective of this experiment was to determine the intensity of immune response to the SARS-CoV-2 virus antigen in the blood of volunteers after their immunization with different variants of the developed agent.

Healthy volunteers 18-60 years of age were included in the trial. All participants of the trial were divided into several groups.
1) Agent for inducing specific immunity against severe acute respiratory syndrome virus SARS-CoV-2, in lyophilized (freeze-dried) form, which contains a single active component, comprising the expression vector based on the genome of the recombinant strain of human adenovirus serotype 26, wherein the E1 and E3 regions are deleted and the ORF6-Ad26 region is replaced by ORF6-Ad5, with an integrated expression cassette selected from SEQ ID NO:1, 10¹¹ viral particles/dose, 9 individuals.
2) Agent for inducing specific immunity against severe acute respiratory syndrome virus

SARS-CoV-2, in lyophilized (freeze-dried) form, which contains a single active component, comprising the expression vector based on the genome of the recombinant strain of human adenovirus serotype 5, wherein the E1 and E3 regions are deleted and the ORF6-Ad26 region is replaced by ORF6-Ad5, with an integrated expression cassette selected from SEQ ID NO:1, 10¹¹ viral particles/dose, 9 individuals.

The volunteers were immunized via a single-dose intramuscular administration of the relevant agent.

Water for injections in an amount of 1.0 ml was added to the vial containing the developed agent in lyophilized form. Then, the vial was shaken up until the lyophilizate was fully dissolved. The developed agent was administered intramuscularly in the deltoid muscle (upper third of the outer aspect of the upper arm). In case where it was impossible to make injection in the deltoid muscle, the agent was injected in the lateral vastus muscle.

Prior to immunization and at days 14 and 28 after immunization, blood samples were collected from the subjects; the mononuclear cells were separated from the samples by density gradient centrifugation in Ficoll solution (1.077 g/mL; PanEco). Then, the separated cells were stained with fluorescent dye CFSE (Invivogen, USA) and placed in the wells of 96-well plate (2*10⁵ cell/well). As a next step, the lymphocytes were re-stimulated in vitro by adding the coronavirus S protein to the culture medium (final protein concentration—1 μg/ml). Intact cells without added antigen were used as a negative control. The percentage of proliferating cells was measured 72 hours following the antigen addition, and the culture medium was sampled for measuring gamma-interferon.

For determining % of proliferating cells, they were stained with the antibodies against marker molecules of T lymphocytes CD3, CD4, CD8 (anti-CD3 Pe-Cy7 (BD Biosciences, clone SK7), anti-CD4 APC (BD Biosciences, clone SK3), anti-CD8 PerCP-Cy5.5 (BD Biosciences, clone SK1)). Proliferating cells (with a lower amount of CFSE dye) CD4+ and CD8+ T lymphocytes were determined in the cell mixture, using high-performance cytofluorometer BD FACS AriaIII (BD Biosciences, USA). The resulting percentage of proliferating cells in each specimen was determined by subtracting the result obtained in the analysis of intact cells from the result obtained in the analysis of cells re-stimulated by the coronavirus S antigen. The findings are shown on FIGS. 3 and 4.

The results of the performed study demonstrated that the intensity of cell-mediated immunity induced by the immunization of volunteers with different variants of the agent (based on the median numbers of proliferating CD4+ and CD8+ T lymphocytes) was increasing as more days passed since the date of the immunization. In all groups, the peak values of proliferating CD4+ and CD8+ T lymphocytes were recorded at day 28 after the immunization. The largest statistically significant difference in the values of proliferating CD4+ and CD8+ T lymphocytes was reported between their values at day 0 and day 28 of the study, p<0.001.

Thus, based on the above findings a conclusion can be made that the immunization with the developed agent is capable to induce the formation of intense antigen-specific cell-mediated anti-infection immunity which is proven by a high level of statistic significance in the measured parameters prior and following the immunization.

### Example 10. Assessment of Adverse Events in Volunteers after a Single- and Double-Shot Immunization by Variants of the Developed Agent

The objective of this experiment was to determine side effects in volunteers following their immunization by different variants of the developed agent.

Healthy volunteers 18-60 years of age were included in the trial. All participants of the trial were divided into several groups.

1) A single-shot intramuscular administration of the agent based on the recombinant human adenovirus serotype 26 (Ad26-too-CMV-S-CoV2) in lyophilized (freeze-dried) form, 10¹¹ viral particles/dose, 9 individuals.

2) A single-shot intramuscular administration of the agent based on the recombinant human adenovirus serotype 5 (Ad5-too-CMV-S-CoV2) in lyophilized (freeze-dried) form, 10¹¹ viral particles/dose, 9 individuals.

3) A double-shot immunization regimen, wherein at first the agent based on the recombinant human adenovirus serotype 26 (Ad26-too-CMV-S-CoV2) in lyophilized (freeze-dried) form, 10¹¹ viral particles/dose, is administered, and 21 days later the agent based on the recombinant human adenovirus serotype 5 (Ad5-too-CMV-S-CoV2) in lyophilized (freeze-dried) form, 10¹¹ viral particles/dose, is administered, 20 individuals.

Table 4 includes data on the most common adverse events reported from the beginning of the trial through the visit (phone call) at Day 180 within the trial.

TABLE 4
Most common adverse events observed after a single-shot administration
of the developed agent in comparison with a double-shot administration

| | Number of Subjects (%) Number of Events | | |
|---|---|---|---|
| | 26 | 5 | 26 + 5 |
| Laboratory and instrumental data | | | |
| Decrease in natural killer cell count | 4 (44.44%)6 | 3 (33.33%) 4 | 4 (20.00%) 6 |
| Increase in T lymphocyte count | 2 (22.22%) 2 | 4 (44.44%) 4 | 10 (50.00%) 10 |
| Increase in CD4 lymphocyte count | 1 (11.11%) 1 | 4 (44.44%) 4 | 8 (40.00%) 8 |
| Increase in B lymphocyte count | 0 (0.00%) 0 | 0 (0.00%) 0 | 7 (35.00%) 7 |
| Increase in lymphocyte count | 2 (22.22%) 2 | 0 (0.00%) 0 | 6 (30.00%) 7 |
| Increase in CD8 lymphocyte count | 1 (11.11%) 1 | 0 (0.00%) 0 | 6 (30.00%) 6 |
| Increase in immunoglobulin E (IgE) | 0 (0.00%) 0 | 0 (0.00%) 0 | 4 (20.00%) 4 |
| level in the blood | | | |
| Increase in immunoglobulin A level | 0 (0.00%) 0 | 1 (11.11%) 1 | 0 (0.00%) 0 |
| Decrease in CD4/CD8 ratio | 1 (11.11%) 1 | 0 (0.00%) 0 | 2 (10.00%) 2 |
| Increase in aspartate aminotransferase | 0 (0.00%) 0 | 0 (0.00%) 0 | 1 (5.00%) 1 |
| level | | | |
| Increase in bilirubin level in the | 0 (0.00%) 0 | 0 (0.00%) 0 | 1 (5.00%) 1 |
| blood | | | |
| Increase in cholesterol level in the | 0 (0.00%) 0 | 0 (0.00%) 0 | 1 (5.00%) 1 |
| blood | | | |
| Increase in natural killer cell count | 0 (0.00%) 0 | 0 (0.00%) 0 | 1 (5.00%) 1 |
| Decrease in immunoglobulin E (IgE) | 0 (0.00%) 0 | 0 (0.00%) 0 | 1 (5.00%) 1 |
| level in the blood | | | |
| Decrease in creatinine level in the | 0 (0.00%) 0 | 0 (0.00%) 0 | 1 (5.00%) 1 |
| blood | | | |
| Decrease in lactate dehydrogenase | 0 (0.00%) 0 | 0 (0.00%) 0 | 1 (5.00%) 1 |
| level in the blood | | | |
| General health disorders and reactions | | | |
| at the site of administration | | | |
| Pain at the vaccination site | 5 (55.56%) 5 | 7 (77.78%) 7 | 12 (60.00%) 19 |
| Pain | 1 (11.11%) 1 | 2 (22.22%) 2 | 6 (30.00%) 9 |
| Induration at the injection site | 2 (22.22%) 2 | 1 (11.11%) 1 | 0 (0.00%) 0 |
| Hyperthermia | 1 (11.11%) 1 | 1 (11.11%) 1 | 5 (25.00%) 5 |
| Pyrexia | 0 (0.00%) 0 | 2 (22.22%) 2 | 0 (0.00%) 0 |
| Asthenia | 0 (0.00%) 0 | 0 (0.00%) 0 | 4 (20.00%) 4 |
| Increased skin temperature at the | 0 (0.00%) 0 | 1 (11.11%) 1 | 0 (0.00%) 0 |
| vaccination site | | | |
| Nervous system disorders | | | |
| Headache | 3 (33.33%) 3 | 4 (44.44%) 4 | 5 (25.00%) 6 |

As demonstrated by the presented data, the incidence of side effects after a single-shot regimen of immunization with the developed agent for inducing specific immunity against severe acute respiratory syndrome virus SARS-CoV-2, in lyophilized (freeze-dried) from, was significantly lower as compared with a double-shot immunization regimen.

### Example 11. Assessment of the Efficacy of Intranasal Immunization with the Developed Agent Based on the Evaluation of Humoral Immune Response

The objective of this study was to verify the efficacy of the developed agent after is intranasal administration.

C57/B16 female mice, 18-20 g, were used in the experiment, 5 animals/group. The following animal groups were formed:
1) A single-dose intranasal administration of the agent based on the recombinant human adenovirus serotype 26 (Ad26-too-CMV-S-CoV2), in lyophilized (freeze-dried) form, 5*10¹⁰ viral particles/dose.
2) A single-dose intranasal administration of the agent based on the recombinant human adenovirus serotype 5 (Ad5-too-CMV-S-CoV2) in lyophilized (freeze-dried) form, 5*10¹⁰ viral particles/dose.
3) A single-dose intranasal administration of the agent based on the recombinant simian adenovirus serotype 25 (simAd25-too-CMV-S-CoV2) in lyophilized (freeze-dried) form, 5*10¹⁰ viral particles/dose.
4) A single-dose intranasal administration of the agent based on the recombinant human adenovirus serotype 26 (Ad26-too-CMV-S-CoV2), in lyophilized (freeze-dried) form, 5*10¹¹ viral particles/dose.
5) A single-dose intranasal administration of the agent based on the recombinant human adenovirus serotype 5 (Ad5-too-CMV-S-CoV2) in lyophilized (freeze-dried) form, 5*10¹¹ viral particles/dose.
6) A single-dose intranasal administration of the agent based on the recombinant simian adenovirus serotype 25 (simAd25-too-CMV-S-CoV2) in lyophilized (freeze-dried) form, 5*10¹¹ viral particles/dose.
7) A single-dose intranasal administration of the buffer solution (negative control).

Three weeks later, blood samples were taken from the tail vein of the animals, and the blood serum was separated. An enzyme-linked immunosorbent assay (ELISA) was used to measure antibody titers according to the following protocol:
1) Antigen was adsorbed onto wells of a 96-well ELISA plate for 16 hours at a temperature of +4° C.
2) Then, for preventing a non-specific binding, the plate was “blocked” with 5% milk dissolved in TPBS in an amount of 100 μl per well. It was incubated in shaker at 37° C. for one hour.
3) Serum samples from the immunized mice were diluted 100-fold, and then a two-fold dilution series was prepared.
4) 50 μl of each of the diluted serum samples were added to the plate wells.
5) Then, incubation at 37° C. for 1 hour was performed.
6) After incubation the wells were washed three times with phosphate buffer.
7) Then, the secondary antibodies against mouse immunoglobulins conjugated with horseradish peroxidase were added.
8) Next, incubation at 37° C. for 1 hour was performed.
9) After incubation the wells were washed three times with phosphate buffer.
10) Then, tetramethylbenzidine (TMB) solution was added which is used as a substrate for horseradish peroxidase and is converted into a colored compound by the reaction. The reaction was stopped after 15 minutes by adding sulfuric acid. Next, using a spectrophotometer, the optical density (OD) of the solution was measured in each well at a wavelength of 450 nm.

Antibody titer was determined as the last dilution at which the optical density of the solution was significantly higher than in the negative control group. The obtained results (geometric mean) are presented in Table 5.

TABLE 5
Antibody titers against SARS-CoV-2 S protein in the blood
serum of mice (geometric mean of antibody titers)

| Animal group | Antibody titer |
|---|---|
| Ad26-too-CMV-S-CoV2, 5*10¹⁰ v.p./dose | 1056 |
| Ad5-too-CMV-S-CoV2, 5*10¹⁰ v.p./dose | 7352 |
| simAd25-too-CMV-S-CoV2, 5*10¹⁰ v.p./dose | 5572 |
| Ad26-too-CMV-S-CoV2, 5*10¹¹ v.p./dose | 2111 |
| Ad5-too-CMV-S-CoV2, 5*10¹¹ v.p./dose | 16890 |
| simAd25-too-CMV-S-CoV2, 5*10¹¹ v.p./dose | 11143 |
| Buffer solution | 0 |

As shown by the experimental results, the intranasal immunization of animals with the developed agent resulted in an increase in antibody titers against the S protein of SARS-CoV-2. Thus, the results of this experiment prove that the developed agent, in lyophilized (freeze-dried) form, administered by intranasal route can be used for inducing specific immunity against severe acute respiratory syndrome virus SARS-CoV-2.

### Example 12. Assessment of the Immunogenicity of the Developed Agent after the Concomitant Intramuscular and Intranasal Immunization

The objective of this study was to verify the efficacy of the developed agent after the concomitant intramuscular and intranasal immunization.

C57/B16 female mice, 18-20 g, were used in the experiment, 5 animals/group. The following animal groups were formed:

1) Simultaneous intranasal administration of the agent based on the recombinant human adenovirus serotype 26 (Ad26-too-CMV-S-CoV2), in lyophilized (freeze-dried) form, 5*10¹⁰ viral particles/dose, and intramuscular administration of the agent based on the recombinant human adenovirus serotype 26 (Ad26-too-CMV-S-CoV2), in lyophilized (freeze-dried) form, 5*10¹⁰ viral particles/dose

2) Intranasal administration of the agent based on the recombinant human adenovirus serotype 26 (Ad26-too-CMV-S-CoV2), in lyophilized (freeze-dried) form, 5*10¹⁰ viral particles/dose

3) Intramuscular administration of the agent based on the recombinant human adenovirus serotype 26 (Ad26-too-CMV-S-CoV2), in lyophilized (freeze-dried) form, 5*10¹⁰ viral particles/dose

4) Simultaneous intranasal administration of the agent based on the recombinant human adenovirus serotype 5 (Ad5-too-CMV-S-CoV2) in lyophilized (freeze-dried) form, 5*10¹⁰ viral particles/dose, and intramuscular administration of the agent based on the recombinant human adenovirus serotype 5 (Ad5-too-CMV-S-CoV2) in lyophilized (freeze-dried) form, 5*10¹⁰ viral particles/dose

5) Intranasal administration of the agent based on the recombinant human adenovirus serotype 5 (Ad5-too-CMV-S-CoV2) in lyophilized (freeze-dried) form, 5*10¹⁰ viral particles/dose

6) Intramuscular administration of the agent based on the recombinant human adenovirus serotype 5 (Ad5-too-CMV-S-CoV2) in lyophilized (freeze-dried) form, 5*10¹⁰ viral particles/dose

7) Simultaneous intranasal administration of the agent based on the recombinant simian adenovirus serotype 25 (simAd25-too-CMV-S-CoV2) in lyophilized (freeze-dried) form, 5*10¹⁰ viral particles/dose, and intramuscular administration of the agent based on the recombinant simian adenovirus serotype 25 (simAd25-too-CMV-S-CoV2) in lyophilized (freeze-dried) form, 5*10¹⁰ viral particles/dose

8) Intranasal administration of the agent based on the recombinant simian adenovirus serotype 25 (simAd25-too-CMV-S-CoV2) in lyophilized (freeze-dried) form, 5*10¹⁰ viral particles/dose

9) Intramuscular administration of the agent based on the recombinant simian adenovirus serotype 25 (simAd25-too-CMV-S-CoV2) in lyophilized (freeze-dried) form, 5*10¹⁰ viral particles/dose

10) Simultaneous intranasal administration of the agent based on the recombinant human adenovirus serotype 26 (Ad26-too-CMV-S-CoV2), in lyophilized (freeze-dried) form, 5*10¹¹ viral particles/dose, and intramuscular administration of the agent based on the recombinant human adenovirus serotype 26 (Ad26-too-CMV-S-CoV2), in lyophilized (freeze-dried) form, 5*10¹¹ viral particles/dose

11) Intranasal administration of the agent based on the recombinant human adenovirus serotype 26 (Ad26-too-CMV-S-CoV2), in lyophilized (freeze-dried) form, 5*10¹¹ viral particles/dose

12) Intramuscular administration of the agent based on the recombinant human adenovirus serotype 26 (Ad26-too-CMV-S-CoV2), in lyophilized (freeze-dried) form, 5*10¹¹ viral particles/dose

13) Simultaneous intranasal administration of the agent based on the recombinant human adenovirus serotype 5 (Ad5-too-CMV-S-CoV2) in lyophilized (freeze-dried) form, 5*10¹¹ viral particles/dose, and intramuscular administration of the agent based on the recombinant human adenovirus serotype 5 (Ad5-too-CMV-S-CoV2) in lyophilized (freeze-dried) form, 5*10¹¹ viral particles/dose

14) Intranasal administration of the agent based on the recombinant human adenovirus serotype 5 (Ad5-too-CMV-S-CoV2) in lyophilized (freeze-dried) form, 5*10¹¹ viral particles/dose

15) Intramuscular administration of the agent based on the recombinant human adenovirus serotype 5 (Ad5-too-CMV-S-CoV2) in lyophilized (freeze-dried) form, 5*10¹¹ viral particles/dose

16) Simultaneous intranasal administration of the agent based on the recombinant simian adenovirus serotype 25 (simAd25-too-CMV-S-CoV2) in lyophilized (freeze-dried) form, 5*10¹¹ viral particles/dose, and intramuscular administration of the agent based on the recombinant simian adenovirus serotype 25 (simAd25-too-CMV-S-CoV2) in lyophilized (freeze-dried) form, 5*10¹¹ viral particles/dose

17) Intranasal administration of the agent based on the recombinant simian adenovirus serotype 25 (simAd25-too-CMV-S-CoV2) in lyophilized (freeze-dried) form, 5*10¹¹ viral particles/dose

18) Intramuscular administration of the agent based on the recombinant simian adenovirus serotype 25 (simAd25-too-CMV-S-CoV2) in lyophilized (freeze-dried) form, 5*10¹¹ viral particles/dose

19) Simultaneous intranasal administration of the buffer solution and intramuscular administration of the buffer solution (negative control)

20) Intranasal administration of the buffer solution (negative control)

21) Intramuscular administration of the buffer solution (negative control)

Three weeks later, blood samples were taken from the tail vein of the animals, and the blood serum was separated. An enzyme-linked immunosorbent assay (ELISA) was used to measure antibody titers according to the following protocol:
1) Antigen was adsorbed onto wells of a 96-well ELISA plate for 16 hours at a temperature of +4° C.
2) Then, for preventing a non-specific binding, the plate was “blocked” with 5% milk dissolved in TPBS in an amount of 100 μl per well. It was incubated in shaker at 37° C. for one hour.
3) Serum samples from the immunized mice were diluted 100-fold and then a two-fold dilution series was prepared.
4) 50 μl of each of the diluted serum samples were added to the plate wells.
5) Then, incubation at 37° C. for 1 hour was performed.
6) After incubation the wells were washed three times with phosphate buffer.
7) Then, the secondary antibodies against mouse immunoglobulins conjugated with horseradish peroxidase were added.
8) Next, incubation at 37° C. for 1 hour was performed.
9) After incubation the wells were washed three times with phosphate buffer.
10) Then, tetramethylbenzidine (TMB) solution was added which was used as a substrate for horseradish peroxidase and was converted into a colored compound by the reaction. The reaction was stopped after 15 minutes by adding sulfuric acid. Next, using a spectrophotometer, the optical density (OD) of the solution was measured in each well at a wavelength of 450 nm.

Antibody titer was defined as the last dilution at which the optical density of the solution was significantly higher than in the negative control group. The obtained results (geometric mean) are presented in Table 6.

TABLE 6
Antibody titers against SARS-CoV-2 S protein in the blood
serum of mice (geometric mean of antibody titers)

| | Animal group | Antibody titer |
|---|---|---|
| 1 | Ad26-too-CMV-S-CoV2, 5*10¹⁰ v.p./dose IN, | 2786 |
| | Ad26-too-CMV-S-CoV2, 5*10¹⁰ v.p./dose IM | |
| 2 | Ad26-too-CMV-S-CoV2, 5*10¹⁰ v.p./dose IN | 919 |
| 3 | Ad26-too-CMV-S-CoV2, 5*10¹⁰ v.p./dose IM | 2111 |
| 4 | Ad5-too-CMV-S-CoV2, 5*10¹⁰ v.p./dose IN, | 33779 |
| | Ad5-too-CMV-S-CoV2, 5*10¹⁰ v.p./dose IM | |
| 5 | Ad5-too-CMV-S-CoV2, 5*10¹⁰ v.p./dose IN | 8445 |
| 6 | Ad5-too-CMV-S-CoV2, 5*10¹⁰ v.p./dose IM | 38802 |
| 7 | simAd25-too-CMV-S-CoV2, 5*10¹⁰ v.p./dose IN, | 19401 |
| | simAd25-too-CMV-S-CoV2, 5*10¹⁰ v.p./dose IM | |
| 8 | simAd25-too-CMV-S-CoV2, 5*10¹⁰ v.p./dose IN | 7352 |
| 9 | simAd25-too-CMV-S-CoV2, 5*10¹⁰ v.p./dose IM | 19401 |
| 10 | Ad5-too-CMV-S-CoV2, 5*10¹⁰ v.p./dose IN, | 44572 |
| | Ad26-too-CMV-S-CoV2, 5*10¹⁰ v.p./dose IM | |
| 11 | Ad26-too-CMV-S-CoV2, 5*10¹⁰ v.p./dose IN, | 51200 |
| | Ad5-too-CMV-S-CoV2, 5*10¹⁰ v.p./dose IM | |
| 12 | Ad5-too-CMV-S-CoV2, 5*10¹⁰ v.p./dose IN, | 58813 |
| | simAd25-too-CMV-S-CoV2, 5*10¹⁰ v.p./dose IM | |
| 13 | simAd25-too-CMV-S-CoV2, 5*10¹⁰ v.p./dose IN, | 51200 |
| | Ad5-too-CMV-S-CoV2, 5*10¹⁰ v.p./dose IM | |
| 14 | Ad26-too-CMV-S-CoV2, 5*10¹⁰ v.p./dose IN, | 22286 |
| | simAd25-too-CMV-S-CoV2, 5*10¹⁰ v.p./dose IM | |
| 15 | simAd25-too-CMV-S-CoV2, 5*10¹⁰ v.p./dose IN, | 25600 |
| | Ad26-too-CMV-S-CoV2, 5*10¹⁰ v.p./dose IM | |
| 16 | Ad26-too-CMV-S-CoV2, 5*10¹¹ v.p./dose IN, | 3676 |
| | Ad26-too-CMV-S-CoV2, 5*10¹¹ v.p./dose IM | |
| 17 | Ad26-too-CMV-S-CoV2, 5*10¹¹ v.p./dose IN, | 1056 |
| 18 | Ad26-too-CMV-S-CoV2, 5*10¹¹ v.p./dose IM | 2786 |
| 19 | Ad5-too-CMV-S-CoV2, 5*10¹¹ v.p./dose IN, | 44572 |
| | Ad5-too-CMV-S-CoV2, 5*10¹¹ v.p./dose IM | |
| 20 | Ad5-too-CMV-S-CoV2, 5*10¹¹ v.p./dose IN | 11143 |
| 21 | Ad5-too-CMV-S-CoV2, 5*10¹¹ v.p./dose IM | 33779 |
| 22 | simAd25-too-CMV-S-CoV2, 5*10¹¹ v.p./dose IN, | 22286 |
| | simAd25-too-CMV-S-CoV2, 5*10¹¹ v.p./dose IM | |
| 23 | simAd25-too-CMV-S-CoV2, 5*10¹¹ v.p./dose IN | 6400 |
| 24 | simAd25-too-CMV-S-CoV2, 5*10¹¹ v.p./dose IM | 19401 |
| 25 | Ad5-too-CMV-S-CoV2, 5*10¹¹ v.p./dose IN, | 51200 |
| | Ad26-too-CMV-S-CoV2, 5*10¹¹ v.p./dose IM | |
| 26 | Ad26-too-CMV-S-CoV2, 5*10¹¹ v.p./dose IN, | 51200 |
| | Ad5-too-CMV-S-CoV2, 5*10¹¹ v.p./dose IM | |
| 27 | Ad5-too-CMV-S-CoV2, 5*10¹¹ v.p./dose IN, | 58813 |
| | simAd25-too-CMV-S-CoV2, 5*10¹¹ v.p./dose IM | |
| 28 | simAd25-too-CMV-S-CoV2, 5*10¹¹ v.p./dose IN, | 51200 |
| | Ad5-too-CMV-S-CoV2, 5*10¹¹ v.p./dose IM | |
| 29 | Ad26-too-CMV-S-CoV2, 5*10¹¹ v.p./dose IN, | 25600 |
| | simAd25-too-CMV-S-CoV2, 5*10¹¹ v.p./dose IM | |
| 30 | simAd25-too-CMV-S-CoV2, 5*10¹¹ v.p./dose IN, | 22286 |
| | Ad26-too-CMV-S-CoV2, 5*10¹¹ v.p./dose IM | |
| 31 | Buffer solution IN | 0 |
| | Buffer solution IM | |
| 32 | Buffer solution IN | 0 |
| 33 | Buffer solution IM | 0 |

As shown by the obtained results, the concomitant intranasal and intramuscular immunization of animals with the developed agent induced a stronger humoral immune response as compared with the immunization via a single administration route. Thus, the results of this experiment prove that the developed agent can be used for inducing specific immunity against the SARS-CoV-2 virus via concomitant and simultaneous intramuscular and intranasal administration.

### INDUSTRIAL APPLICABILITY

All the provided examples prove the efficacy of the pharmaceutical agents ensuring the effective induction of immune response against the SARS-CoV-2 virus and the industrial applicability.

1. An agent for inducing specific immunity against severe acute respiratory syndrome virus SARS-CoV-2, the agent being in lyophilized (freeze-dried) form, the agent comprising:
a single active component, comprising an expression vector comprising a genome of recombinant strain of human adenovirus serotype 26, wherein the E1 and E3 regions are deleted and the ORF6-Ad26 region is replaced by ORF6-Ad5, with an integrated expression cassette selected from SEQ ID NO:1, SEQ ID NO:2, or SEQ ID NO:3.

2. An agent for inducing specific immunity against severe acute respiratory syndrome virus SARS-CoV-2, the agent being in lyophilized (freeze-dried) form, the agent comprising:
a single active component, comprising an expression vector comprising a genome of the recombinant strain of human adenovirus serotype 5, wherein the E1 and E3 regions are deleted, with an integrated expression cassette selected from SEQ ID NO:1, SEQ ID NO:2, or SEQ ID NO:3.

3. An agent for inducing specific immunity against severe acute respiratory syndrome virus SARS-CoV-2, the agent being in lyophilized (freeze-dried) form, the agent, comprising:
a single active component, comprising an expression vector comprising a genome of the recombinant strain of simian adenovirus serotype 25, wherein the E1 and E3 regions are deleted, with an integrated expression cassette selected from SEQ ID NO:4, SEQ ID NO:2, or SEQ ID NO:3.

4. The agent of claim 1, wherein the lyophilized (freeze-dried) form is reconstituted in a buffer solution comprising, by mass %:

| | |
|---|---|
| tris | from 0.0180 to 0.0338 |
| sodium chloride | from 0.1044 to 0.1957 |
| sucrose | from 5.4688 to 10.2539 |
| magnesium chloride hexahydrate | from 0.0015 to 0.0028 |
| EDTA | from 0.0003 to 0.0005 |
| polysorbate-80 | from 0.0037 to 0.0070 |
| water | the remaining part. |

5. A method of inducing immune response against the SARS-CoV-2 virus, the method comprising intranasal or intramuscular administration, or concomitant intranasal and intramuscular administration of the agent of claim 1.

6. The method of claim 5, wherein the agent for intranasal administration is at a dose of 5*10¹⁰-5*10¹¹ viral particles.

7. The method of claim 5 wherein the agent for intramuscular administration is at a dose of 5*10¹⁰-5*10¹¹ viral particles.

8. The method of claim 5 wherein for the concomitant intranasal and intramuscular administration, the agent is administered intramuscularly at a dose of 5*10¹⁰-5*10¹¹ viral particles and intranasally at a dose of 5*10¹⁰-5*10¹¹ viral particles.

9. The agent of claim 2, wherein the lyophilized (freeze-dried) form is reconstituted in a buffer solution comprising, by mass %:

| | |
|---|---|
| tris | from 0.0180 to 0.0338 |
| sodium chloride | from 0.1044 to 0.1957 |
| sucrose | from 5.4688 to 10.2539 |
| magnesium chloride hexahydrate | from 0.0015 to 0.0028 |
| EDTA | from 0.0003 to 0.0005 |
| polysorbate-80 | from 0.0037 to 0.0070 |
| water | the remaining part. |

10. The agent of claim 3, wherein the lyophilized (freeze-dried) form is reconstituted in a buffer solution comprising, by mass %:

| | |
|---|---|
| tris | from 0.0180 to 0.0338 |
| sodium chloride | from 0.1044 to 0.1957 |
| sucrose | from 5.4688 to 10.2539 |
| magnesium chloride hexahydrate | from 0.0015 to 0.0028 |
| EDTA | from 0.0003 to 0.0005 |
| polysorbate-80 | from 0.0037 to 0.0070 |
| water | the remaining part. |

11. A method of inducing immune response against the SARS-CoV-2 virus, the method comprising intranasal or intramuscular administration, or concomitant intranasal and intramuscular administration of the agent of claim 2.

12. The method of claim 11, wherein the agent for intranasal administration is at a dose of 5*10¹⁰-5*10¹¹ viral particles.

13. The method of claim 11, wherein the agent for intramuscular administration is at a dose of 5*10¹⁰-5*10¹¹ viral particles.

14. The method of claim 11, wherein for the concomitant intranasal and intramuscular administration, the agent is administered intramuscularly at a dose of 5*10¹⁰-5*10¹¹ viral particles and intranasally at a dose of 5*10¹⁰-5*10¹¹ viral particles.

15. A method of inducing immune response against the SARS-CoV-2 virus, the method comprising intranasal or intramuscular administration, or concomitant intranasal and intramuscular administration of the agent of claim 3.

16. The method of claim 15, wherein the agent for intranasal administration is at a dose of 5*10¹⁰-5*10¹¹ viral particles.

17. The method of claim 15, wherein the agent for intramuscular administration is at a dose of 5*10¹⁰-5*10¹¹ viral particles.

18. The method of claim 15, wherein for the concomitant intranasal and intramuscular administration, the agent is administered intramuscularly at a dose of 5*10¹⁰-5*10¹¹ viral particles and intranasally at a dose of 5*10¹⁰-5*10¹¹ viral particles.
